(19) United States
(12) Patent Application Publication
(10) Pub. No.: US 2022/0162663 A1
(43) Pub. Date: May 26, 2022

(54) LABEL-FREE DETECTION OF MYCOBACTERIA USING SURFACE ENHANCED RAMAN SPECTROSCOPY

(71) Applicants: Agency for Science, Technology and Research, Singapore (SG); Matralix Pte Ltd, Singapore (SG)

(72) Inventors: Malini Olivo, Singapore (SG); Jayakumar Perumal, Singapore (SG); Renuga Gopal, Singapore (SG); Saif Abdul Kadir Khan, Singapore (SG)

(21) Appl. No.: 17/602,271

(22) PCT Filed: Apr. 8, 2020
(86) PCT No.: PCT/SG2020/050215

§ 371 Date: Oct. 7, 2021

(30) Foreign Application Priority Data
Apr. 9, 2019 (SG) 10201903159V

(51) Int. Cl. C12Q 1/02; G01N 21/65; G01N 1/40

(52) U.S. Cl. CPC C12Q 1/02; G01N 21/658; G01N 2001/4061; G01N 2800/26; G01N 1/4055

(57) ABSTRACT

A method of identifying a Mycobacterium using surface enhanced Raman spectroscopy (SERS), disclosed herein, comprises disposing a sample suspected to comprise a Mycobacterium on a SERS-active substrate, detecting surface enhanced Raman signals corresponding to an alpha-mycolic acid, a methoxy-mycolic acid, and a keto-mycolic acid from the sample disposed on the SERS-active substrate, and determining one or more ratios of intensity of the surface enhanced Raman signals to identify the Mycobacterium. Disclosed herein also includes a method of detecting a mycobacterial disease using surface enhanced Raman spectroscopy (SERS), the method comprising identifying a Mycobacterium according to the method described above, and determining the mycobacterial disease based on the Mycobacterium identified.

### CROSS-REFERENCE TO RELATED APPLICATION

This application claims the benefit of priority of Singapore Patent Application No. 10201903159V, filed 9 Apr. 2019, the content of it being hereby incorporated by reference in its entirety for all purposes.

### TECHNICAL FIELD

The present disclosure relates to a method of identifying a Mycobacterium, and a method of detecting a mycobacterial disease, both methods using surface enhanced Raman spectroscopy (SERS).

### BACKGROUND

Mycolic acids are high molecular weight fatty acids having about 60 to 90 carbon atoms in their fatty acid chains, wherein the fatty acid chains include an α-branched alkyl chain and a β-hydroxy chain. Mycolic acids may be found in the cell walls of a group of bacteria, such as mycobacteria tuberculosis and nontuberculous mycobacteria (NTM).

Mycolic acids may be comprised of three forms. The three forms may include an alpha-mycolic acid (AMA) having a cyclopropane, a keto-mycolic acid (KMA) having an epoxy functional group, and a methoxy-mycolic acid (MMA) having an ester functional group. These mycolic acids have a role in protecting bacteria from dehydration and chemical damage, helping bacteria to survive harsh conditions. For example, the mycolic acids may be present in a mycobacterial cell wall, wherein the mycolic acids may be covalently bonded or closely associated with arabinogalactan polymers, a structural component of the mycobacterial cell wall, to maintain rigidity of the cell wall. The mycolic acids also behave as a shield, protecting the mycobacteria from lysozymes and oxygen radicals produced from phagocytosis, and the shield even helps the mycobacteria to survive in low pH (acidic environment). As a Mycobacterium may contain a mycolic acid, methods have been developed to detect a mycolic acid for determining the absence or presence of a Mycobacterium.

Methods used to detect mycolic acid may include high performance liquid chromatography (HPLC), thin layer chromatography, smear microscopy and mass spectrometry. In HPLC, detection for mycolic acid in a sample may be through the observation of a HPLC spectral profile and analysis of a sample's retention time in the HPLC column. The methods used, however, suffer from one or more limitations.

For example, methods such as smear microscopy and mass spectrometry, may involve large machines that cannot be reduced into smaller point-of-care devices. An example of a point-of-care device may be a portable mycolic acid detection kit. The methods mentioned above may be too time consuming for detecting multiple mycolic acids, or even for a single mycolic acid. The methods may also require a mycolic acid to be present at a sufficient concentration for the mycolic acid to be even detectable from a sample, which may in turn require the culturing of a sample suspected to contain a Mycobacterium. Moreover, the methods may require use of one or more specific reagents which render the methods economically undesirable.

In addition, the methods may suffer from detection interference if lipids or other biological materials do not get isolated from the sample suspected to contain mycolic acid. To extract mycolic acid from a bacteria sample, the methods may require the bacteria sample to be subjected to an additional ball milling step. The ball milling step is meant to break down the cell wall to release mycolic acid therefrom. At the same time, lipids and other proteins may be released. Such lipids and proteins may interfere with the detection of the mycolic acid in such methods. Hence, the methods mentioned above tend to require additional extraction and/or isolation procedures.

There is thus a need to provide for a solution that addresses one or more of the limitations mentioned above. The solution should at least provide for a method of identifying the bacteria, e.g. Mycobacterium, and not just identifying the presence or absence of a single mycolic acid.

### SUMMARY

In a first aspect, there is provided for a method of identifying a Mycobacterium using surface enhanced Raman spectroscopy (SERS), the method comprising:

disposing a sample suspected to comprise a Mycobacterium on a SERS-active substrate;

detecting surface enhanced Raman signals corresponding to an alpha-mycolic acid, a methoxy-mycolic acid, and a keto-mycolic acid from the sample disposed on the SERS-active substrate; and

determining one or more ratios of intensity of the surface enhanced Raman signals to identify the Mycobacterium.

A method of detecting a mycobacterial disease using surface enhanced Raman spectroscopy (SERS), the method comprising:

identifying a Mycobacterium according to the method described in various embodiments of the first aspect; and

determining the mycobacterial disease based on the Mycobacterium identified.

### BRIEF DESCRIPTION OF THE DRAWINGS

The drawings are not necessarily to scale, emphasis instead generally being placed upon illustrating the principles of the invention. In the following description, various embodiments of the present disclosure are described with reference to the following drawings, in which:

FIG. 1A is a field emission scanning electron microscopy (FESEM) image of silver coated silicon nanopillars. The scale bar denotes 1 μm.

FIG. 1B is a FESEM image of 60 nm gold colloids. The scale bar denotes 1 μm.

FIG. 2A shows a surface enhanced Raman spectroscopy (SERS) spectrum of alpha-mycolic acid (AMA). The alpha-mycolic acid was synthetically obtained, and the spectrum is an average of 100 AMA SERS spectra.

FIG. 2B shows a SERS spectrum of methoxy-mycolic acid (MMA). The methoxy-mycolic acid was synthetically obtained, and the spectrum is an average of 100 MMA SERS spectra. p FIG. 2C shows a SERS spectrum of keto-mycolic acid (KMA). The keto-mycolic acid was synthetically obtained, and the spectrum is an average of 100 KMA SERS spectra.

FIG. 3A shows a SERS spectrum obtained from mycolic acids present in a delipidated mycobacterial (DL-MTB) sample. The spectrum is an average of 10 SERS spectra.

FIG. 3B shows a SERS spectrum obtained from mycolic acids present in a non-delipidated mycobacterial (NDL-MTB) sample. The spectrum is an average of 10 SERS spectra.

FIG. 4 compares a SERS spectrum obtained from a DL-MTB sample (top plot) with a SERS spectrum of AMA that was synthetically obtained (bottom plot). The SERS spectrum for AMA is an average of 100 SERS spectra. The SERS spectrum for DL-MTB is an average of 10 SERS spectra. B and D are used as references to denote two batches of spectral data for producing the average spectrum of the DL-MTB.

FIG. 5 compares a SERS spectrum obtained from a DL-MTB sample (top plot) with a SERS spectrum of KMA that was synthetically obtained (bottom plot). The SERS spectrum for KMA is an average of 100 SERS spectra. The SERS spectrum for DL-MTB is an average of 10 SERS spectra. B and D are used as references to denote two batches of spectral data for producing the average spectrum of the DL-MTB.

FIG. 6 shows the identification and matching of SERS peaks corresponding to those of AMA (top plot) and KMA (bottom plot) in a SERS spectrum obtained from a DL-MTB sample. Both SERS spectra are an average of 10 SERS spectra. B and D are used as references to denote two batches of spectral data for producing the average spectrum of the DL-MTB.

### DETAILED DESCRIPTION

The following detailed description refers to the accompanying drawings that show, by way of illustration, specific details and embodiments in which the invention may be practised.

Features that are described in the context of an embodiment may correspondingly be applicable to the same or similar features in the other embodiments.

Features that are described in the context of an embodiment may correspondingly be applicable to the other embodiments, even if not explicitly described in these other embodiments. Furthermore, additions and/or combinations and/or alternatives as described for a feature in the context of an embodiment may correspondingly be applicable to the same or similar feature in the other embodiments.

Various embodiments of the first aspect relate to a method of identifying a Mycobacterium, from a sample, using surface enhanced Raman spectroscopy (SERS).

Mycobacteria may contain mycolic acids. Examples of mycolic acids that may be found in a Mycobacterium include alpha-mycolic acid, keto-mycolic acid, and methoxy-mycolic acid. In the method of the first aspect, mycolic acids, including these three mycolic acids, may be identified through their SERS signals, as each of these mycolic acids produces a specific signature in a SERS spectrum.

In the method of the first aspect, the amount of one mycolic acid present in a sample may be compared to another mycolic acid present therein to obtain a ratio. The present method establishes such a ratio by comparing the SERS signal intensity of one mycolic acid to another, and one or more such ratios may be determined depending on the number of types of mycolic acids present therein. The SERS signal intensity of a mycolic acid depends on its amount in the sample, i.e. higher amount of a mycolic acid produces a higher SERS signal intensity and lower amount leads to lower SERS signal intensity. For example, a sample subjected to the method of the first aspect may produce SERS signal intensities for alpha-mycolic acid, keto-mycolic acid, and methoxy-mycolic acid. One or more ratios of the SERS signal intensity between alpha-mycolic acid to keto-mycolic, alpha-mycolic acid to methoxy-mycolic acid, and/or keto-mycolic acid to methoxy-mycolic acid, may be determined. The determination of such ratios in the method of the first aspect helps identify the Mycobacterium that may be present in a sample, as different Mycobacterium may contain different mycolic acids in different amounts. If the ratios indicate a higher amount of methoxy-mycolic acid, pathogenic tuberculosis causing Mycobacterium may be identified. Through the method of the first aspect, a method of detecting a mycobacterial disease using surface enhanced Raman spectroscopy (SERS) is also developed, which various embodiments disclosed herein relate to. Said differently, the mycobacterial disease detected is based on the Mycobacterium identified through the method of the first aspect.

The method of the first aspect includes a step of disposing a sample suspected to contain a Mycobacterium on a SERS-active substrate. The term “SERS-active substrate” herein refers to a material capable of enhancing Raman scattering. The sample may be brought into contact with or disposed in the proximity of the SERS-active substrate to produce one or more SERS signals corresponding to the one or more mycolic acids that may be present therein. In other words, the present method does not require the use of a label (e.g. a binding entity) to attach mycolic acids to the SERS-active substrate for generating SERS signals. Conversely, in some mycobacteria detection methods, a suspected sample has to be mixed with a binding entity (a protein, a ligand, an antibody, etc.) that specifically targets a mycolic acid. The mycolic acid bound to the binding entity is then able to attach to a substrate for producing a signal to indicate the presence of a mycolic acid. In other methods, the substrate has to be immobilized with the binding entity for capturing specifically a mycolic acid for detection. Further, some methods use a binding entity that gives off a signal when it gets attached to a mycolic acid, to indicate presence of a mycolic acid. The present method detects and identifies various mycolic acids without the use of such labels, and hence may be referred herein as a “label-free” method.

The method of the first aspect uses SERS, which is advantageous for identifying a Mycobacterium. SERS allows for single molecule detection, i.e. to identify the presence and/or absence of a mycolic acid, due to its high sensitivity and reproducibility. As the method of the first aspect has high sensitivity, it may detect for mycolic acids present at concentrations having a magnitude in the range of nM to μM. As the method of the first aspect is able to detect mycolic acids at such low concentrations, the culturing of a sample to increase the amount of mycolic acids for detection may be avoided. The method of the first aspect minimizes the steps needed to prepare a sample and requires neither the reagents nor time consuming use of complicated machines associated with detection techniques like HPLC, chromatography, mass spectrometry, etc. The detection and identification of mycobacteria, through the method of the first aspect, can then be carried out more rapidly, reducing a user's period of exposure to harmful mycobacteria.

The method of the first aspect, through use of SERS, is rendered non-invasive, i.e. may be carried out in vitro and/or ex vivo, and does not require destruction of a sample. SERS renders the method of the first aspect a high spatial resolution technique and allows for a sample to be in a form of solid, liquid or gas for analysis. The SERS signal intensities and their ratios are also easily and quickly quantifiable for identifying different mycobacteria (e.g. within a day). Nevertheless, the method of the first aspect may be used in conjunction with other existing detection techniques to ascertain the presence or absence of mycobacteria.

Details of various embodiments of the method of the first aspect and the method of detecting a mycobacterial disease based on the method of the first aspect, and advantages associated with the various embodiments, are now described below.

The method of the first aspect provides for identifying a Mycobacterium using surface enhanced Raman spectroscopy (SERS). The method may comprise disposing a sample suspected to comprise a Mycobacterium on a SERS-active substrate, detecting surface enhanced Raman signals corresponding to an alpha-mycolic acid, a methoxy-mycolic acid, and a keto-mycolic acid from the sample disposed on the SERS-active substrate, and determining one or more ratios of intensity of the surface enhanced Raman signals to identify the Mycobacterium.

In embodiments where the sample is a liquid or converted into a liquid, the sample may be disposed on the SERS-active substrate or mixed with the SERS-active substrate. In other words, disposing the sample on the SERS-active substrate may comprise providing the sample in the form of a liquid. To provide the sample in the form of a liquid may comprise mixing the sample in a solvent. Thus, disposing the sample on the SERS-active substrate may comprise mixing the sample in a solvent before disposing the sample on the SERS-active substrate.

The solvent may comprise chloroform and methanol. This combination of chloroform and methanol may allow for different lipids to be dissolved therein, which renders easier extraction of lipids from the membrane of a Mycobacterium. In embodiments where chloroform and methanol are used, the chloroform and methanol may be present in a volume ratio of 1:1 to 9:1, 2:1 to 9:1, 3:1 to 9:1, 4:1 to 9:1, 5:1 to 9:1, 6:1 to 9:1, 7:1 to 9:1, 8:1 to 9:1. These ratios demonstrated for dissolution of different lipids in the absence of excessive amounts of both chloroform and methanol, thereby rendering for efficient extraction of lipids.

The method of the first aspect is advantageously versatile in that extraction of mycolic acids from a Mycobacterium may or may not be needed to generate the one or more SERS signals corresponding to the mycolic acids, as a mycolic acid need not be in physical contact with a SERS-active substrate to generate a SERS signal. This is because a mycolic acid in the proximity of the SERS-active substrate sufficiently close to render an interaction between the mycolic acid and SERS-active substrate generates the SERS signal. For example, a surface of a Mycobacterium's membrane may happen to be in close proximity (e.g. 10 nm or less) with the SERS-active substrate, and this may be sufficient to generate a plasmonic enhancement to give off a Raman signal. With this, a sample suspected to comprise the Mycobacterium may not need to be subjected to an extraction process to extract the alpha-mycolic acid, the methoxy-mycolic acid, and the keto-mycolic acid from the Mycobacterium suspected to be comprised in the sample. This reduces the number of steps and reagents needed for identification of the mycolic acids and Mycobacterium to be carried out more rapidly. Nevertheless, if preferred, the method of the first aspect may further comprise extracting the alpha-mycolic acid, the methoxy-mycolic acid, and the keto-mycolic acid from the Mycobacterium suspected to be comprised in the sample prior to disposing the sample on the SERS-active substrate. Extracting of the alpha-mycolic acid, the methoxy-mycolic acid, and the keto-mycolic acid may comprise disintegrating a cell wall of the Mycobacterium suspected to be comprised in the sample, as mycolic acids are structural components in the cell wall of a Mycobacterium.

To disintegrate the cell wall, extracting the alpha-mycolic acid, the methoxy-mycolic acid, and the keto-mycolic acid may comprise subjecting the sample to saponification, mechanical milling, delipidating, or a combination thereof. Other suitable means for releasing mycolic acids from the cell wall may be used.

In various embodiments, extracting the alpha-mycolic acid, the methoxy-mycolic acid, and the keto-mycolic acid may comprise delipidating the Mycobacterium suspected to be comprised in the sample. Using delipidation as an example, clearer SERS signals may be generated as the extracted mycolic acids may be isolated from other lipids released from disintegration of the cell wall. If other lipids happen to be present, the other lipids may generate SERS signals that interfere with the SERS signals of mycolic acids. Hence, the method of the first aspect may further comprise removing lipids from the sample prior to disposing the sample on the SERS-active substrate. Notwithstanding this, as the method of the first aspect has high sensitivity and accuracy, the SERS signals of mycolic acids may be detected and identified even in the presence of other lipids.

In various embodiments, the delipidating may comprise inactivating the Mycobacterium suspected to be comprised in the sample, and separating the sample into an aqueous phase and an organic phase. The organic phase provides for dissolution of the other lipids released from a Mycobacterium, which may be removed to isolate the mycolic acids in the aqueous phase.

The inactivating of the Mycobacterium may comprise incubating the sample at a temperature ranging from 20° C. to 40° C., 20° C. to 30° C., or 30° C. to 40° C. Incubation of the sample may be carried out for at least 24 hours. Alternatively, the inactivation of a Mycobacterium may be carried out by exposing the Mycobacterium to gamma irradiation. For example, a sample suspected to comprise a Mycobacterium may be inactivated by exposing to 2.4 mRads of ionizing gamma irradiation using a ¹³⁷Cs source. Inactivation may be confirmed by an Alamar Blue assay technique.

To reduce ion suppression by phospholipids and hydrolyzed fatty acids (i.e. incomplete extraction of mycolic acids due to interference from other phospholipids and fatty acids present in the membrane of a Mycobacterium), a second delipidation may be carried out, which may further comprise mixing the aqueous phase with one or more organic solvents to form a mixture, separating the mixture into a further aqueous phase, a further organic phase and an intermediate phase, wherein the intermediate phase may comprises the delipidated Mycobacterium suspected to be comprised in the sample. In various embodiments, the one or more organic solvents may comprise chloroform.

After obtaining the intermediate phase, the method of the first aspect may further comprise drying the intermediate phase to obtain the delipidated Mycobacterium suspected to be comprised in the sample.

The method of the first aspect may further comprise contacting the delipidated Mycobacterium suspected to be comprised in the sample with a mixture of alcohol and a base, heating the resultant mixture, and adding an extraction solvent and an acid to the heated mixture. This may assist in the break down of a Mycobacterium's membrane for selectively extraction of mycolic acids.

In various embodiments, the extraction solvent may comprise chloroform. In various embodiments, the acid may comprise hydrochloric acid. In various embodiments, the base may comprise potassium hydroxide. In various embodiments, the alcohol may comprise methanol. Other suitable extraction solvents, acid, base and alcohol that do not alter or destroy the mycolic acids may be used.

The method of the first aspect uses an SERS-active substrate to generate SERS signal corresponding to a mycolic acid. The SERS-active substrate may be coated with or formed entirely of a SERS-active material, such as, but no limited to, noble metals such as silver, palladium, gold, platinum, iridium, rhodium, ruthenium, or an alloy thereof. Other SERS-active material may include copper, aluminum, or an alloy thereof, wherein the alloy may include noble metal. The SERS-active substrate may comprise or consist of gold, silver, or an alloy thereof. The SERS-active substrate may comprise nanoparticles of gold, silver, an alloy thereof, or a mixture thereof. Alternatively, the SERS-active substrate may comprise a layer of gold, silver, or an alloy thereof coated on a support. In certain instances, the SERS-active substrate may be formed from a non-SERS-active material, such as plastic, ceramic, composites, glass or organic polymers, and coated with a SERS-active material mentioned above to render plasmonic characteristics.

The SERS-active substrate may be a support comprising at least one SERS-active nanostructure disposed on a surface of the support. The support comprising at least one SERS-active nanostructure disposed on a surface of the support may be one of a dielectric support, a semiconductor support, or a paper support, the support comprising at least one nanostructure may be coated with a layer of a SERS-active material. The SERS-active material may comprise a layer of gold, silver, or an alloy thereof. The term “nanostructure” herein refers to a material having at least one dimension that is in the nanometer range. At least one dimension of the nanostructure may be 1000 nm or less, or ranging from 100 nm to 1000 nm. Examples of a nanostructure may include, but are not limited to, nano-islands, nanopillars, nanoflakes, nanoparticles, and a combination thereof. For example, the support may comprise a silicon substrate having an array of nanostructures disposed thereon. The nanostructures may comprise silicon nanopillars. As another example, the support comprising at least one SERS-active nanostructure disposed on a surface of the support may be a dielectric support, such as a glass support or a silica-coated silicon support. The at least one SERS-active nanostructure disposed on a surface of the dielectric support may be in the form of a plurality of nanopillars coated with a layer of the SERS-active material. The plurality of nanopillars may have a height ranging from 250 nm to 300 nm, 260 nm to 300 nm, 270 nm to 300 nm, 280 nm to 300 nm, 290 nm to 300 nm, etc. The spacing between each of the nanopillars may be 100 nm or less, 90 nm or less, 80 nm or less, 70 nm or less, 60 nm or less, 50 nm or less, 40 nm or less, 30 nm or less, 20 nm or less, 10 nm or less, etc.

In various embodiments, detecting the surface enhanced Raman signals may comprise exposing the sample disposed on the SERS-active substrate to an electromagnetic radiation and generating one or more surface enhanced Raman signals from the sample. The electromagnetic radiation may have a wavelength ranging from 100 nm to 1000 nm, 200 nm to 1000 nm, 300 nm to 1000 nm, 400 nm to 1000 nm, 500 nm to 1000 nm, 600 nm to 1000 nm, 700 nm to 1000 nm, 800 nm to 1000 nm, or 900 nm to 1000 nm. Wavelengths of these ranges, including the longer wavelengths, may render stronger signals from a sample. In some embodiments, the electromagnetic radiation my have a wavelength of 785 nm.

From the method of the first aspect, the one or more surface enhanced Raman signals generated may be in the range of 600 cm⁻¹ to 1800 cm⁻¹. SERS signals in this range may correspond to one or more of the three mycolic acids mentioned above.

In various embodiments, detecting the surface enhanced Raman signals may comprise comparing the one or more surface enhanced Raman signals generated from the sample with the surface enhanced Raman signals generated from an artificially synthesized alpha-mycolic acid, an artificially synthesized methoxy-mycolic acid, and an artificially synthesized keto-mycolic acid. Detecting of the surface enhanced Raman signals may comprise determining presence of and/or intensity of surface enhanced Raman signals corresponding to the alpha-mycolic acid, the methoxy-mycolic acid, and the keto-mycolic acid. By comparing SERS signals obtained from the present method with the SERS signals of artificially synthesized mycolic acids, the mycolic acids present in a sample may be identified.

After identifying the presence and/or absence of mycolic acids, determining the one or more ratios of intensity of the surface enhanced Raman signals may be carried out. This may comprise correlating the intensity of the surface enhanced Raman signals corresponding to the alpha-mycolic acid, the methoxy-mycolic acid, and the keto-mycolic acid to a concentration for each of the alpha-mycolic acid, the methoxy-mycolic acid, and the keto-mycolic acid detected in the sample, and comparing the concentrations to obtain the one or more ratios for identifying the Mycobacterium. As already mentioned above, different Mycobacterium contains different amounts of each mycolic acids. By comparing the ratios of SERS signal intensity to establish the composition of the mycolic acids in a sample, the Mycobacterium may be identified.

Non-limiting examples of the Mycobacterium that may be identified through the method of the first aspect include a non-tuberculous Mycobacterium or a species of Mycobacterium tuberculosis complex. The non-tuberculous Mycobacterium may comprise Mycobacterium avium complex, Mycobacterium kansasii, or Mycobacterium abscessus. The species of Mycobacterium tuberculosis complex may comprise Mycobacterium tuberculosis, Mycobacterium africanum, Mycobacterium bovis, or Mycobacterium microti.

The sample suspected to comprise Mycobacterium may contain (i) Mycobacterium, and/or (ii) any of the alpha-mycolic acid, the methoxy-mycolic acid, and the keto-mycolic acid, present in a concentration ranging from 1 nM to 1 μM. The method of the first aspect has high sensitivity and allows for detection and identification of Mycobacterium in such concentrations.

The method of the first aspect is non-invasive as it may be carried out in vitro or ex vivo, through the use of SERS, for identifying a Mycobacterium in sample. By identifying the Mycobacterium in a sample, the method of the first aspect may be used to detect a mycobacterial disease. The mycobacterial disease may comprise a pulmonary disease, a lymphatic disease, or a skin disease. The mycobacterial disease may also comprise tuberculosis.

As already mentioned above, the present disclosure relates to a method of detecting a mycobacterial disease using surface enhanced Raman spectroscopy (SERS). The method may be based on various embodiments of the first aspect. Embodiments and advantages described for the method of the first aspect can be analogously valid for the present method of detecting a mycobacterial disease described herein, and vice versa. As the various embodiments and advantages have already been described above and in the examples demonstrated herein, they shall not be iterated for brevity.

The present method of detecting a mycobacterial disease using surface enhanced Raman spectroscopy (SERS) may comprise identifying a Mycobacterium according to various embodiments described for the method of the first aspect, and determining the mycobacterial disease based on the Mycobacterium identified.

The mycobacterial disease may comprise a pulmonary disease, a lymphatic disease, or a skin disease. The mycobacterial disease may also comprise tuberculosis.

The present method of detecting a mycobacterial disease is non-invasive, as it may be carried out in vitro or ex vivo, through the use of SERS and based on the method of the first aspect.

The word “substantially” does not exclude “completely” e.g. a composition which is “substantially free” from Y may be completely free from Y. Where necessary, the word “substantially” may be omitted from the definition of the invention.

In the context of various embodiments, the articles “a”, “an” and “the” as used with regard to a feature or element include a reference to one or more of the features or elements.

In the context of various embodiments, the term “about” or “approximately” as applied to a numeric value encompasses the exact value and a reasonable variance.

As used herein, the term “and/or” includes any and all combinations of one or more of the associated listed items.

Unless specified otherwise, the terms “comprising” and “comprise”, and grammatical variants thereof, are intended to represent “open” or “inclusive” language such that they include recited elements but also permit inclusion of additional, unrecited elements.

### EXAMPLES

The present disclosure relates to a method based on surface enhanced Raman spectroscopy (SERS) for identifying a Mycobacterium, such as a tuberculosis (TB) bacterium. The present method is able to identify a Mycobacterium through quantifying the SERS signals corresponding to mycolic acids present or absent in a sample. In other words, the mycolic acids act as biomarkers for identification of the Mycobacterium.

The present method has improved sensitivity from using SERS to detect mycolic acids in a sample without the use of any labels. That is to say, a sample suspected to contain a Mycobacterium, from which the mycolic acids are retrieved, need not be mixed with any binding entity that specifically targets a mycolic acid for binding to a SERS-active substrate to produce the SERS signals. Hence, the present method is termed herein a “label-free” method.

The present method incorporates use of a SERS-active substrate. The SERS-active substrate may be termed herein a “SERS scattering platform”. SERS signals are produced when a mycolic acid gets in contact with or in the proximity of the SERS-active substrate. The SERS signals may be detected in the form of one or more spectra from the SERS-active substrate. The SERS-active substrate may be a substrate comprising silver coated silicon nanopillars (AgSNP) or gold nanoparticles.

In the present method, the SERS signals obtained from mycolic acids in a sample may be statistically analysed. For example, the SERS signal for each mycolic acids may be identified from a sample. The intensity of the SERS signals for each mycolic acid may be compared to obtain one or more ratios of the intensity of the SERS signals, which may provide information regarding the amount of each mycolic acid present and absent in the sample, and their relative ratios. This robust and efficient analytical protocol leads to accurate quantification of mycolic acids extracted from a sample, which may then be used to identify the Mycobacterium in the sample.

In addition, the present method is feasible for identifying the mycolic acids present and absent in a gamma irradiated bacteria, and without the need to first extract the mycolic acids from the bacteria cell wall. The gamma irradiation renders a sample safer to work with, as it inactivates a Mycobacterium suspected to be contained in the sample.

Advantageously, as demonstrated in the examples below, the present method may be used to identify a Mycobacterium even when the mycolic acids happen to be present in a sample at a concentration magnitude of nM to μM.

The present methods of identifying a Mycobacterium and detecting a mycobacterial disease are discussed in details, by way of non-limiting examples, as set forth below.

### Example 1A: Preparation of Silicon Nanopillar SERS-Active Substrate

For fabrication of silicon nanopillar (SNP) SERS-active substrate, silicon etching was performed using Inductively-Coupled Plasma Reactive Ion etch (ICP-RIE) system from Oxford Instruments. Undoped or P type silicon wafer may be used for SNP fabrication.

As a first step, a silicon wafer was subjected to oxygen plasma treatment using O₂ gas under 10⁻¹⁵ mTorr chamber pressure for 5 mins to 10 mins based on the requirement. This step was carried out in order to increase the silica layer on the silicon surface.

In the second step, a combination of SF₆:O₂ gas was used in a ratio of 1.1 to 1.21 at the etch rate of 2.5 to 2.8 nm/s. Randomly arranged silicon nanopillars with 250 to 300 nm height were obtained, and spacing between the nanopillar was below 100 nm.

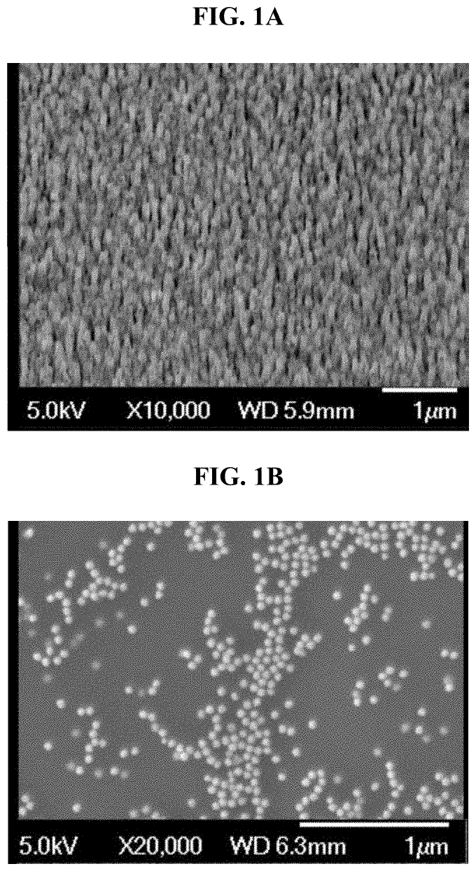

Following this step, either silver only or a combination of gold and silver may be deposited by sputtering or by an electron-beam evaporation system, which involves a physical vapor deposition technique. FIG. 1A shows the silver coated silicon nanopillars, which is just one example of the SERS-active substrate used.

### Example 1B: SERS Spectrum of Mycolic Acids

The SERS spectrum of various mycolic acids were first acquired. The mycolic acids, which include alpha-mycolic acid (AMA), keto-mycolic acid (KMA), and methoxy-mycolic acid (MMA), were purchased from Avanti polar lipids, US. The purchased mycolic acids are synthetically obtained (i.e. artificially produced) mycolic acids. These three mycolic acids were focused on, as they may constitute the more prominent mycolic acids in mycobacteria.

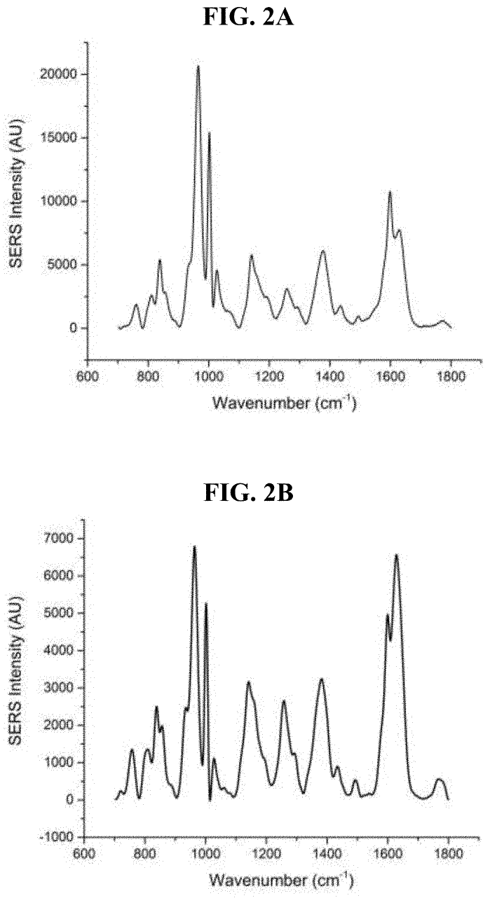

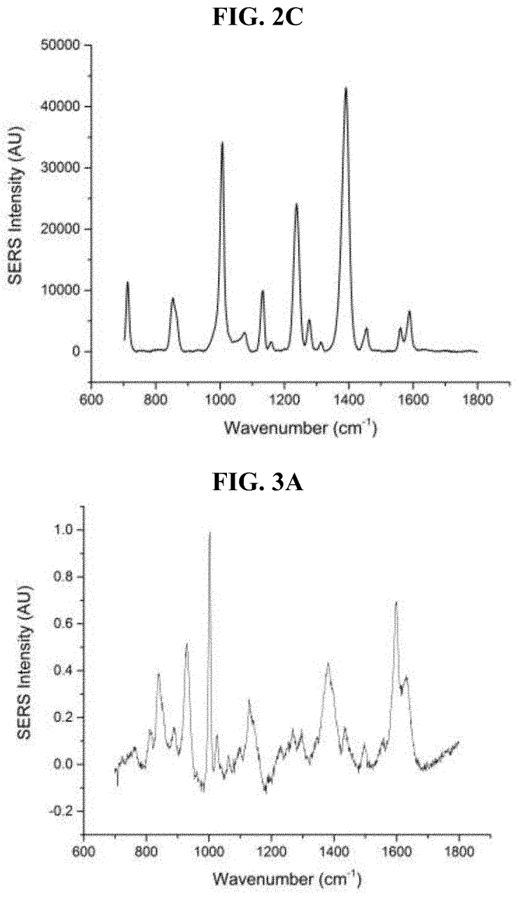

For reproducibility and accuracy, multiple SERS scans were carried out for each of the three mycolic acids to obtain three resultant SERS spectra, respectively, wherein each of the resultant SERS spectrum is an average of the SERS spectra obtained from the multiple scans. Hence, the resultant spectrum may be termed herein an “average spectrum” or “average SERS spectrum”. FIG. 2A to 2C respectively depict the average SERS spectrum for alpha-mycolic acid, keto-mycolic acid, and methoxy-mycolic acid. To generate the SERS signals that form the SERS spectrum, the SERS-active substrate used may either be silver coated silicon nanopillars (FIG. 1A) or gold nanoparticles (FIG. 1B).

In the present method, the SERS signal intensity of a mycolic acid depends on its amount present in the sample. For example, in FIG. 2A, alpha-mycolic acid may have a signature peak near 1000 cm⁻¹ and the intensity of this peak may depend on the concentration of the alpha-mycolic acid present, i.e. higher amount of alpha-mycolic acid generates a higher SERS signal intensity of the signature peak and lower amount may lead to lower SERS signal intensity. In the present method, the SERS signal intensity for each mycolic acid may be compared to obtain their relative ratios, and by determining the relative ratios of mycolic acids through their SERS signal intensity, the Mycobacterium present or absent in a sample may be identified. Said differently, through SERS signal intensity, the amount of a mycolic acid relative to another may be determined as set out in the present method for identification of a Mycobacterium from a sample. For instance, all three mycolic acids may be present in both mycobacteria tuberculosis and nontuberculous mycobacteria (NTM) family of bacteria, but in different ratios, and in many instances, pathogenic tuberculosis causing Mycobacterium may have a higher amount of methoxy-mycolic acid. The SERS signal intensities between the three mycolic acids may then be compared to identify the Mycobacterium. Advantageously, the present method is able to identify the Mycobacterium in spite of different Mycobacterium having all three mycolic acids but present in different amounts.

### Example 2: Delipidation and Non-Delipidation of Sample

Mycolic acids are one of the dominant lipids present in the mycobacteria tuberculosis family of bacteria which causes tuberculosis. The present method is versatile in that the characterization of mycolic acids may be carried out with or without extraction of mycolic acids from mycobacteria. In other words, the present method may involve an extraction step for extracting mycolic acids from the mycobacteria. As a non-limiting example, a sample suspected to contain a mycobacteria may be fed to a ball mill and crushed therein before contacting the sample with an SERS-active substrate to obtain the SERS spectra, from which mycolic acids may be identified as present or absent. In such instances, the SERS signals of other lipids may show up in the SERS spectra, as mycolic acids were not isolated. Such a sample containing other lipids is then referred herein as a “non-delipidated” sample. Where mycolic acids have been isolated from other lipids, the sample is then referred herein as a “delipidated” sample.

In various examples, to identify the composition of mycolic acids present in a tuberculosis causing bacterial strain and to compare them with mycolic acids found in a non-tuberculosis causing bacterial strain. Extraction of mycolic acids was carried out. The extraction was carried out based on Matralix extraction protocol, which is discussed below by way of a non-limiting example of extraction by delipidation of a sample suspected to contain a Mycobacterium.

100 mg of bacteria was treated with 1 mL chloroform-methanol (volume ratio of 2:1) in a pyrex glass culture tube and stirred overnight at 40° C. to inactivate the pathogens and extract free lipids. Two blanks containing only 1 mL chloroform-methanol (volume ratio of 2:1) were also included to serve as controls. After overnight incubation, the mixtures were transferred to screw-capped tubes and rinsed once with 500 μL H₂O. Delipidation was carried out by vortexing the mixture thoroughly for 1 minute and centrifuging at 14,000 rpm for 10 minutes at room temperature. The lower organic phase (containing free lipids) was removed. To reduce ion suppression by phospholipids and hydrolyzed fatty acids, a second delipidation with additional 500 μL chloroform was carried out by repeating the above steps for delipidation. The second delipidation may remove free lipids loosely bound to a Mycobacterium's membrane. The upper aqueous phase and the lower organic phase were carefully removed. The intermediate layer was transferred to a fresh tube and dried to obtain the delipidated bacteria.

The delipidated bacteria, having lipids that were loosely bound to a Mycobacterium's membrane removed therefrom, were then subjected to mycolic acids extraction. The extraction of mycolic acids involves separation of mycolic acids from the cell membrane, wherein a base and an alcohol may be used. In brief, 2 mL 20% methanolic KOH was added to 100 mg of the delipidated bacteria cells. Samples were incubated at 80° C. for 30 minutes, then autoclaved at 121° C. for 30 minutes. Chloroform (2 mL) was added, followed by 1.5 mL 50% HCl. Samples were centrifuged at 2,000 rpm at room temperature for 10 minutes. The chloroform layer was collected and air-dried overnight at room temperature to obtain the mycolic acids. Following such steps, the mycolic acids can be used to identify the bacteria as set out in the present method.

In one example, the mycolic acids extracted from a whole native bacteria (without delipidation), and another batch of mycolic acids obtained after delipidation of the outer membrane of a bacterium, were compared.

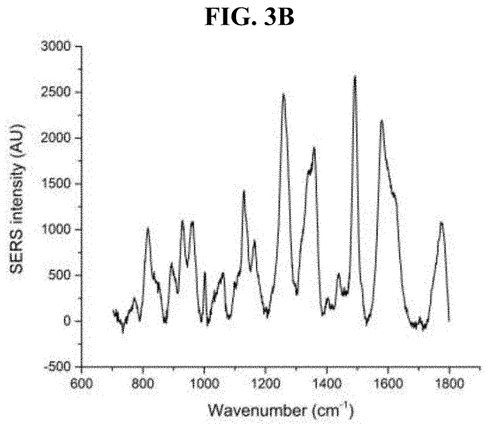

FIG. 3A and 3B respectively show a SERS spectrum of delipidated and non-delipidated sample. From FIG. 3A and 3B, the present method is apparently versatile as the SERS peaks corresponding to alpha-mycolic acid (AMA), keto-mycolic acid (KMA), and methoxy-mycolic acid (MMA), may be observed from both samples. This means that even if other lipid components happen to be present, the different mycolic acids may still be identified through the present method.

### Example 3: Ratio Analysis of SERS Signal Intensities Using Delipidated Bacteria

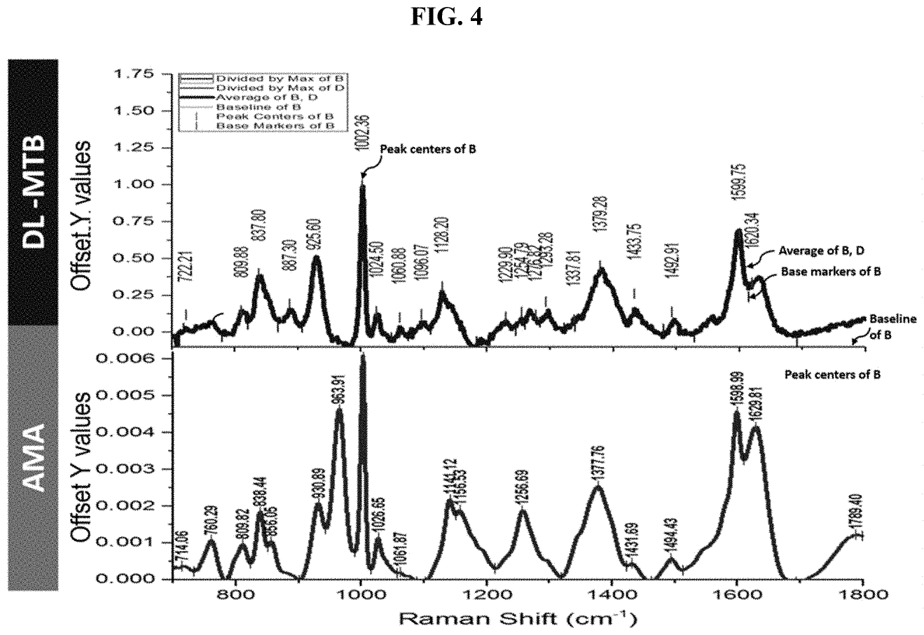

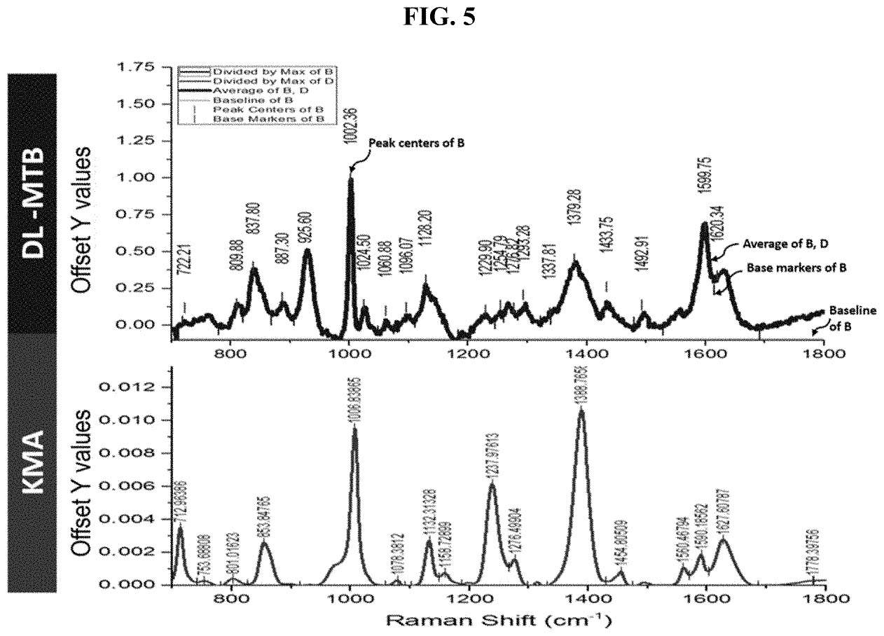

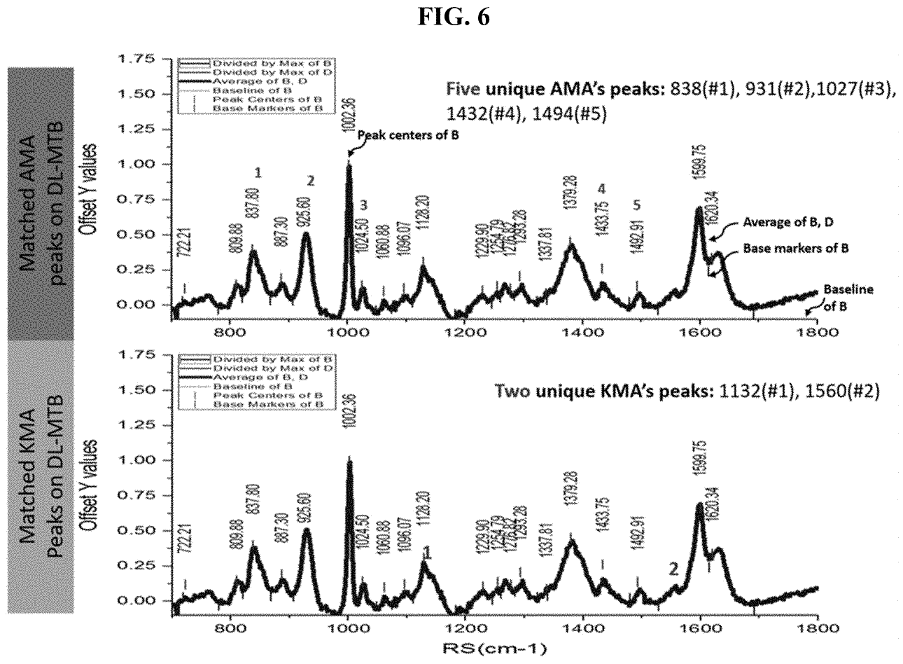

In subsequent studies, delipidated mycolic acids from different bacteria strains were used to demonstrate for the present method. Specifically, the relative concentrations of alpha-mycolic acid (AMA), keto-mycolic acid (KMA), and methoxy-mycolic acid (MMA), were quantified through their SERS signal intensities. For example, in FIG. 4, the specific and representative AMA peaks that may be observed in the DL-MTB spectrum are shown. In FIG. 5, the specific and representative KMA peaks that may be observed in the DL-MTB spectrum are shown. In FIG. 6, the specific and representative AMA and KMA peaks that may be observed in the DL-MTB spectrum are shown. Specifically, in FIG. 6, the exact AMA and KMA peaks that can be seen in the DL-MTB spectrum are indicated. The quantification may be carried out using chemometric and/or advanced machine learning methods. For example, among the three mycolic acids, the SERS signature of alpha-mycolic acid and methoxy-mycolic acid may resemble each other more closely. Hence, a chemometric method may be used to identify relative ratios of the three mycolic acids.

### Example 4: Commercial and Potential Applications

In summary, the present method is a label-free SERS method which is potentially usable for diagnosis of tuberculosis. The present method is able to identify the three different mycolic acids, i.e. alpha-mycolic acid, keto-mycolic acid, and methoxy-mycolic acid, and keto-mycolic acid, and estimate the ratio of each of the mycolic acids present in a sample suspected to contain a Mycobacterium. The ratio in which the three mycolic acids exist relative to each other, which may be established through the SERS signal intensities of the mycolic acids, helps identify pathogenic mycobacterial strains from non-pathogenic ones.

The present method is also compatible for identifying gamma irradiated bacteria without the need to extract mycolic acids. The gamma irradiation helps to inactivate the bacteria, improving bio-safety of the present method, and renders on-site rapid diagnosis of a mycobacterial disease possible.

While the invention has been particularly shown and described with reference to specific embodiments, it should be understood by those skilled in the art that various changes in form and detail may be made therein without departing from the spirit and scope of the invention as defined by the appended claims. The scope of the invention is thus indicated by the appended claims and all changes which come within the meaning and range of equivalency of the claims are therefore intended to be embraced.

1. A method of identifying a Mycobacterium using surface enhanced Raman spectroscopy (SERS), the method comprising:
disposing a sample suspected to comprise a Mycobacterium on a SERS-active substrate;
detecting surface enhanced Raman signals corresponding to an alpha-mycolic acid, a methoxy-mycolic acid, and a keto-mycolic acid from the sample disposed on the SERS-active substrate; and
determining one or more ratios of intensity of the surface enhanced Raman signals to identify the Mycobacterium.

2. The method of claim 1, wherein disposing the sample comprises mixing the sample in a solvent to provide the sample in the form of a liquid, wherein the solvent comprises chloroform and methanol.

3-5. (canceled)

6. The method of claim 1, wherein the sample suspected to comprise the Mycobacterium is not subjected to an extraction process to extract the alpha-mycolic acid, the methoxy-mycolic acid, and the keto-mycolic acid from the Mycobacterium suspected to be comprised in the sample.

7. The method of claim 1, further comprising extracting the alpha-mycolic acid, the methoxy-mycolic acid, and the keto-mycolic acid from the Mycobacterium suspected to be comprised in the sample prior to disposing the sample on the SERS-active substrate.

8. (canceled)

9. The method of claim 7, wherein extracting the alpha-mycolic acid, the methoxy-mycolic acid, and the keto-mycolic acid comprises subjecting the sample to saponification, mechanical milling, delipidating, or a combination thereof.

10. The method of claim 7, further comprising removing lipids from the sample prior to disposing the sample on the SERS-active substrate.

11. (canceled)

12. The method of claim 9, wherein the delipidating comprises:
inactivating the Mycobacterium suspected to be comprised in the sample; and
separating the sample into an aqueous phase and an organic phase.

13.-14 (canceled)

15. The method of claim 12, further comprising:
mixing the aqueous phase with one or more organic solvents to form a mixture;
separating the mixture into a further aqueous phase, a further organic phase and an intermediate phase, wherein the intermediate phase comprises the delipidated Mycobacterium suspected to be comprised in the sample.

16. (canceled)

17. The method of claim 15, further comprising drying the intermediate phase to obtain the delipidated Mycobacterium suspected to be comprised in the sample.

18-20. (canceled)

21. The method of claim 1, wherein disposing the sample on the SERS-active substrate comprises mixing the sample in a solvent before disposing the sample on the SERS-active substrate.

22-23. (canceled)

24. The method of claim 1, wherein the SERS-active substrate is a support comprising at least one SERS-active nanostructure disposed on a surface of the support.

25. The method of claim 24, wherein the support comprising at least one SERS-active nanostructure disposed on a surface of the support is one of a dielectric support, a semiconductor support, or a paper support, the support comprising at least one nanostructure coated with a layer of a SERS-active material.

26. (canceled)

27. The method of claim 25, wherein the support comprises a silicon substrate having an array of nanostructures disposed thereon, wherein the nanostructures comprise silicon nanopillars.

28. (canceled)

29. The method of claim 1, wherein detecting the surface enhanced Raman signals comprises exposing the sample disposed on the SERS-active substrate to an electromagnetic radiation and generating one or more surface enhanced Raman signals from the sample.

30-31. (canceled)

32. The method of claim 29, wherein the one or more surface enhanced Raman signals generated are in the range of 600 cm⁻¹ to 1800 cm⁻¹

33. The method of claim 29, wherein detecting the surface enhanced Raman signals comprises comparing the one or more surface enhanced Raman signals generated from the sample with the surface enhanced Raman signals generated from an artificially synthesized alpha-mycolic acid, an artificially synthesized methoxy-mycolic acid, and an artificially synthesized keto-mycolic acid.

34. (canceled)

35. The method of claim 1, wherein determining the one or more ratios of intensity of the surface enhanced Raman signals comprises:
correlating the intensity of the surface enhanced Raman signals corresponding to the alpha-mycolic acid, the methoxy-mycolic acid, and the keto-mycolic acid to a concentration for each of the alpha-mycolic acid, the methoxy-mycolic acid, and the keto-mycolic acid detected in the sample; and
comparing the concentrations to obtain the one or more ratios for identifying the Mycobacterium.

36-43. (canceled)

44. A method of detecting a mycobacterial disease using surface enhanced Raman spectroscopy (SERS), the method comprising:
identifying a Mycobacterium according to the method of claim 1; and
determining the mycobacterial disease based on the Mycobacterium identified.

45. The method of claim 44, wherein the mycobacterial disease comprises a pulmonary disease, a lymphatic disease, or a skin disease.

46. (canceled)

47. The method of claim 44, wherein the method is carried out in vitro or ex vivo.
